(19) United States
(12) Patent Application Publication
(10) Pub. No.: US 2022/0160962 A1
(43) Pub. Date: May 26, 2022

(54) MULTI-FUNCTIONAL DRIP SYSTEM WITH FUNCTIONS OF DETECTION AND ALERT

(71) Applicant: Jing-Syuan HONG, Taichung (TW)

(72) Inventor: Jing-Syuan HONG, Taichung (TW)

(21) Appl. No.: 16/953,371

(22) Filed: Nov. 20, 2020

(51) Int. Cl. A61M 5/168

(52) U.S. Cl. CPC A61M 5/1689; A61M 2205/18; A61M 2205/3306; A61M 2205/581; A61M 2205/8206; A61M 2205/583

(57) ABSTRACT

A multi-functional drip system with functions of detection and alert contains a flow meter installed in a drip tube which is installed under a liquid supply tube; the liquid supply tube receiving liquid from a drip bag; liquid within the liquid supply tube dropping into the drip tube drop by drop; then the liquid being transferred into a body of a patient through a liquid transfer tube; the flow meter serving to detect a drip amount of the drips passing through the drip tube per unit time; the flow meter transmitting detection data outwards; a transceiver connected to the flow meter for transferring detection data of the flow meter; and a processing unit signally connected to the transceiver for receiving the data from the transceiver; the processing unit further recording the drip detection times per unit time so as to determine the flow speed of the drips.

### FIELD OF THE INVENTION

The present invention is related to drip control, and in particular to a. multi-functional drip system with functions of detection and alert

### BACKGROUND OF THE INVENTION

Drip injection is widely used medically. In drip injection, the drips are firstly filled in the drip bag and then the drips are transferred to a liquid supply tube and then drops into a drip tube below the liquid supply tube and then flows into an injector.

However, in the prior art drip devices, if the drips drop into the drip tube with a fast or lower speed, the flow rate and the effect of the drips will be affected so as to have the problem of the safety of the patient.

In the prior art drip device, the speed of drips cannot be known, generally, it is determined by medical persons, but this way has the problem of low accuracy and it is also time and labor wasted. However, it cannot know when is the time that the drips in the drip tube will be used up. The time is determined by the medical persons to go onsite for viewing the amount of the drops within the bag.

### SUMMARY OF THE INVENTION

Accordingly, the object of the present invention is to provide a multi-functional drip system with functions of detection and alert, wherein the messages about the drip speeds are determined and monitored, which are provided to the medical persons in hospitals so that the medical person may adapt desire processes. The medical persons need not take time to monitor the states of the drip tubes. All the messages are detected automatically and transferred to the monitor persons automatically. Furthermore alarm and alert light are also provided to display to the desired person. All the processes are made automatically so as to save large amount of manpower. Furthermore all the data can be further processed statistically.

To achieve above object, the present invention provides a multi-functional drip system with functions of detection and alert, comprising: a flow meter installed in a drip tube which is installed under a liquid supply tube; the liquid supply tube receiving liquid from a drip bag; liquid within the liquid supply tube dropping into the drip tube drop by drop; then the liquid being transferred into the body of a patient through a liquid transfer tube; the flow meter serving to detect a drip amount of the drips passing through the drip tube per unit time; the flow meter transmitting detect data outwards; a transceiver connected to the flow meter for transferring detection data of the flow meter; and a processing unit signally connected to the transceiver for receiving the data from the transceiver; the processing unit further recording the drip detection times per unit time so as to determine the flow speed of the drips; that is, the flow speed of the drips dropping into the drip tube.

The various objects and advantages of the present invention will be more readily understood from the following detailed description when read in conjunction with the appended drawing.

### BRIEF DESCRIPTION OF THE DRAWINGS

FIG. 1 is a schematic view about the element assembly of the present invention.

FIG. 2 shows an application of the present invention for another element assembly.

FIG. 3 is a block diagram about the element assembly of the present invention.

FIG. 4 is another diagram about another element assembly of the present invention.

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

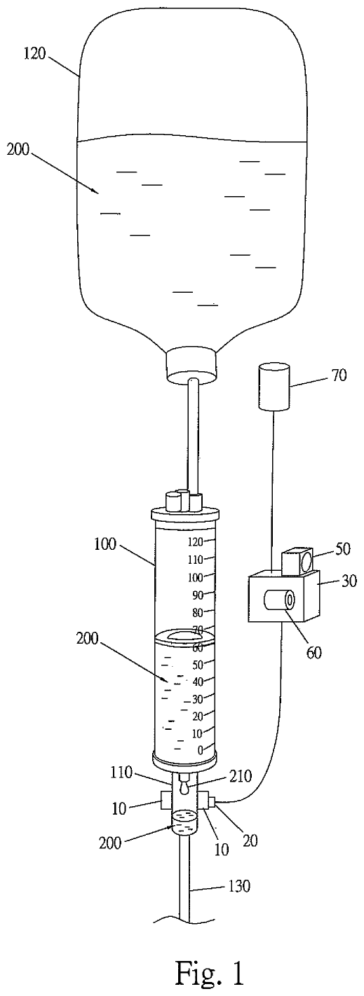

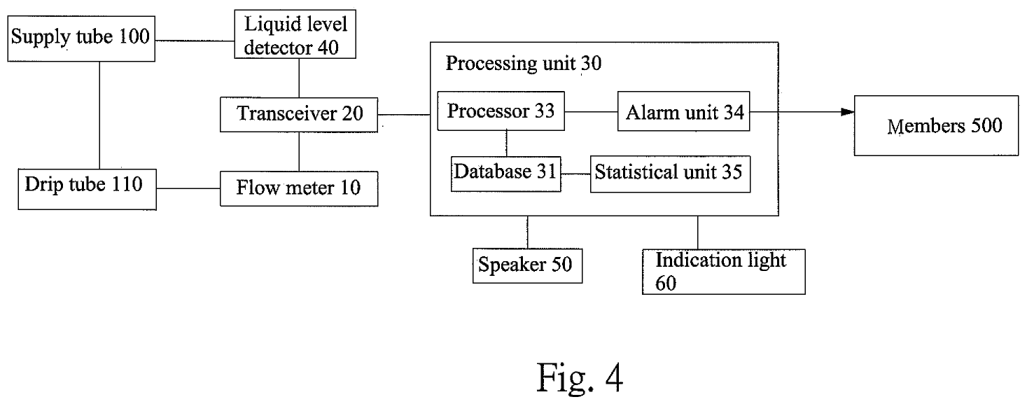

With reference to FIGS. 1 to 4, the structure of the present invention is illustrated.

A flow meter 10 is installed in a drip tube 110 which is installed under a liquid supply tube 100, as illustrated in FIG. 1. The liquid supply tube 100 receives liquid from a drip bag 120. The liquid 200 within the liquid supply tube 100 drops into the drip tube 110 drop by drop. Then the liquid 200 is transferred into the body of a patient through a liquid transfer tube 130. The flow meter 10 serves to detect the drip amount of the drips 210 passing through the drip tube 110 per unit time. The flow meter 10 transmits the detect data outwards. The flow meter 10 is an optical sensor or an infrared sensor.

A transceiver 20 is connected to the flow meter 10 for transferring detection data of the flow meter 10.

A processing unit 30 is signally connected to the transceiver 20 for receiving the data from the transceiver 20. The processing unit 30 further records the drip detection times per unit time so as to determine the flow speed of the drips, that is, the flow speed of the drips dropping into the drip tube 110.

The processing unit 30 may be installed near the drip tube 110 or be installed at a far end of the drip tube 110.

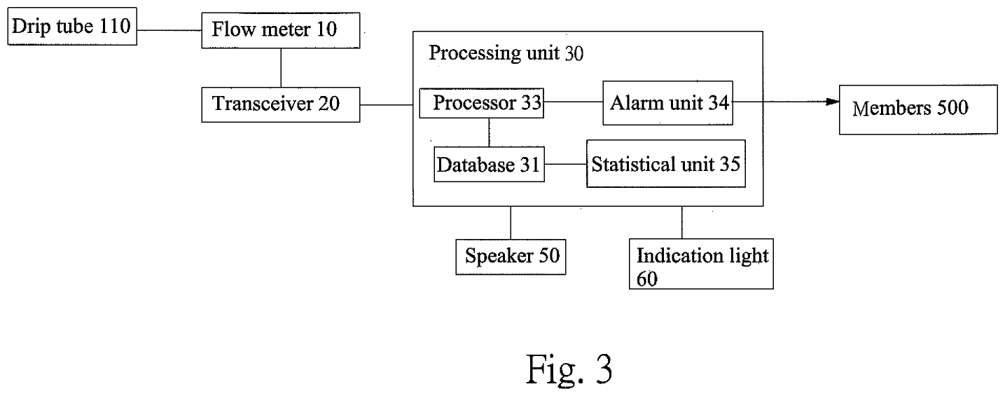

As illustrated in FIG. 3, the processing unit 30 further includes the following elements.

A processor 33 serves to receive the detection signal from the flow meter 10, and calculates the times of detection received per unit time so as to determine a drip speed which is the drip amount per unit time and the drip speed is stored in a database 31.

An alarm unit 34 is connected to the processor 33. When the drip speed is greater than or lower than a preset speed, an alarm signal is transferred outwards. When in a preset time, no drip is detected, that is, the liquid 200 within the liquid supply tube 100 is exhausted or the liquid supply tube 100 is sealed without drips to flow out. Then the alarm unit 34 transfers alarm signals outwards to far end members 500.

A statistical unit 35 is connected to the database 31. The statistical unit 35 makes statistical calculation based on the drip speeds in different time periods and the results are stored or transferred outwards.

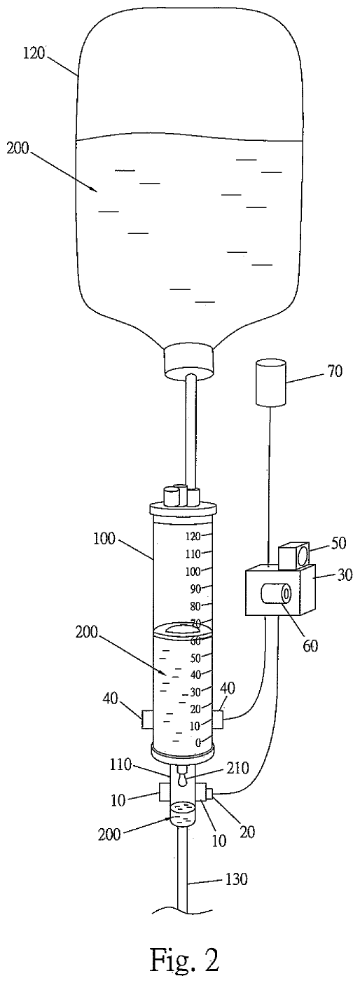

With reference to FIGS. 2 to 4, the present invention further includes a liquid level detector 40 which is connected to the transceiver 20 and is installed at the liquid supply tube 100. The liquid level detector 40 serves to detect levels of liquid 200 of the liquid supply tube 100 and the detect results are transferred to the processing unit 30 through the transceiver 20. When the level is lower than a preset value, the alarm unit 34 transfers alarm signals outwards to alert the medical persons that the liquid 200 of the liquid supply tube 100 is too small or is used up so that the medical person will supply liquid 200 to the liquid supply tube 100 or makes next processes.

In the present invention, the liquid level detector 40 detects the level of the liquid supply tube 100 by laser or other optical detection, however, any way for detecting levels is within the scope of the present invention.

The present invention further includes a speaker 50 which is connected to the processing unit 30. The information of the processing unit 30 is broadcasted through the speaker 50. The speaker 50 also broadcasts alarm messages from the alarm unit 34 to alert medical persons.

An indication light 60 is connected to the processing unit 30. The indication light 60 serves to display the function and states of the flow meter 10, the liquid supply tube 100 and the processing unit 30, alert signals of the alarm unit 34, states about the drip speeds, and states about the levels of the liquid supply tube 100 so as to alert medical persons.

A battery 70 is connected to the processing unit 30 and supplies power to the processing unit 30.

Advantages of the present invention are that: the messages about the drip speeds are determined and monitored, which are provided to the medical persons in hospitals so that the medical person may adapt desire processes. The medical persons need not take time to monitor the states of the drip tubes. All the messages are detected automatically and transferred to the monitor persons automatically. Furthermore alarm and alert light are also provided to display to the desired person. All the processes are made automatically so as to save large amount of manpower. Furthermore all the data can be further processed statistically.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A multi-functional drip system with functions of detection and alert, comprising:
a flow meter installed in a drip tube which is installed under a liquid supply tube; the liquid supply tube receiving liquid from a drip bag; liquid within the liquid supply tube dropping into the drip tube drop by drop; then the liquid being transferred into the body of a patient through a liquid transfer tube; the flow meter serving to detect a drip amount of the drips passing through the drip tube per unit time; the flow meter transmitting detection data outwards;
a transceiver connected to the flow meter for transferring detection data of the flow meter; and
a processing unit signally connected to the transceiver for receiving the data from the transceiver; the processing unit further recording the drip detection times per unit time so as to determine the flow speed of the drips; that is, the flow speed of the drips dropping into the drip tube.

2. The multi-functional drip system with functions of detection and alert as claimed in claim 1, wherein the processing unit is installed near the drip tube or be installed at a far end of the drip tube.

3. The multi-functional drip system with functions of detection and alert as claimed in claim 1, wherein the processing unit further including
a processor for receiving the detection signal from the flow meter, and calculating the times of detection received per unit time so as to determine drip speed which is a drip amount per unit time and the drip speed is stored in a database.

4. The multi-functional drip system with functions of detection and alert as claimed in claim 3, wherein the processing unit further comprises n alarm unit which is connected to the processor; when the drip speed is greater than or lower than a preset speed, an alarm signal is transferred outwards; when in a preset time, no drip is detected, that is, the liquid within the liquid supply tube is exhausted or the liquid supply tube is sealed without drip to flow out; then the alarm unit transfers alarm signals outwards to far end members.

5. The multi-functional drip system with functions of detection and alert as claimed in claim 3, wherein the processing unit further comprises a statistical unit which is connected to the database; the statistical unit makes statistical calculation based on the drip speeds in different time periods and calculation results are stored or transferred outwards.

6. The multi-functional drip system with functions of detection and alert as claimed in claim 1, further comprising a liquid level detector which is connected to the transceiver and is installed at the liquid supply tube; the liquid level detector serves to detect levels of liquid of the liquid supply tube and detect results are transferred to the processing unit through the transceiver; and when the level is lower than a preset value, the alarm unit transfers alarm signals outwards to alert the medical persons that the liquid of the liquid supply tube is too small or is used up so that the medical person supplies liquid to the liquid supply tube or makes next processes.

7. The multi-functional drip system with functions of detection and alert as claimed in claim 6, wherein the liquid level detector detects the level of the liquid supply tube by laser or other optical detection.

8. The multi-functional drip system with functions of detection and alert as claimed in claim 1, further comprising a speaker which is connected to the processing unit; the information of the processing unit is broadcasted through the speaker; the speaker also broadcasts alarm messages from the alarm unit to alert medical persons.

9. The multi-functional drip system with functions of detection and alert as claimed in claim 1, wherein an indication light is connected to the processing unit; the indication light serves to display the function and states of the flow meter, the liquid supply tube and the processing unit, alert signals of the alarm unit, states about the drip speeds, and states about the levels of the liquid supply tube so as to alert medical persons.

10. The multi-functional drip system with functions of detection and alert as claimed in claim 1, wherein a battery is connected to the processing unit and supplies power to the processing unit.

11. The multi-functional drip system with functions of detection and alert as claimed in claim 1, wherein the flow meter is an optical sensor or an infrared sensor.
